(19) United States
(12) Patent Application Publication
(10) Pub. No.: US 2012/0178826 A1
(43) Pub. Date: Jul. 12, 2012

(54) Wound Dressing Compositions Suitable for Use in Negative Pressure Wound Therapy

(76) Inventors: Jarl Jensen, (US); Ravi Ramjit, Floral Park, NY (US)

(21) Appl. No.: 13/344,595

(22) Filed: Jan. 5, 2012

Related U.S. Application Data
(63) Continuation-in-part of application No. 12/387,836, filed on May 7, 2009, now abandoned.
Application No. 12/387,836 is a continuation-in-part of application No. 10/867,388, filed on Jun. 14, 2004, now abandoned.

(51) Int. Cl. A61L 15/24; A61L 15/28; A61L 15/58

(52) U.S. Cl. 514/772.6; 523/105

(57) ABSTRACT

The present invention relates to a wound dressing composition suitable for use in a negative pressure wound therapy application comprising hydrocolloids, hot melt acrylic adhesives, tackifiers, elastomeric binders, and extenders, and if desired, active ingredients.

This application is a Continuation-in-Part of U.S. patent application Ser. No. 12/387,836, filed on May, 7, 2009, which is a Continuation-In-Part of U.S. patent application Ser. No. 10/867,388 filed on Jun. 14, 2004. The entire contents of said applications are hereby incorporated by reference.

### BACKGROUND OF THE INVENTION

A. Field of the Invention

Wound closure involves the inward migration of epithelial and subcutaneous tissue adjacent the wound. This migration is ordinarily assisted through the inflammatory process, whereby blood flow is increased and various functional cell types are activated. Through the inflammatory process, blood flow through damaged or broken vessels is stopped by capillary level occlusion, whereafter cleanup and rebuilding operations may begin. Unfortunately, this process is hampered when a wound is large or has become infected. In such wounds, a zone of stasis (i.e. an area in which localized swelling of tissue restricts the flow of blood to the tissues) forms near the surface of the wound.

Without sufficient blood flow, the epithelial and subcutaneous tissues surrounding the wound not only receive diminished oxygen and nutrients, but are also less able to successfully fight bacterial infection, and, thus are less able to naturally close the wound. Until recently, such difficult wounds were addressed only through the use of sutures or staples. Although still widely practiced and often effective, such mechanical closure techniques suffer a major disadvantage in that they produce tension on the skin tissue adjacent the wound. In particular, the tensile force required in order to achieve closure using sutures or staples causes very high localized stresses at the suture or staple insertion point. These stresses commonly result in the rupture of the tissue at said insertion point, which can eventually cause wound dehiscence and additional tissue loss.

Additionally, some wounds harden and inflame to such a degree, due to infection, that closure by stapling or suturing is not feasible. Wounds not reparable by suturing or stapling generally require prolonged hospitalization, with its attendant high cost, and major surgical procedures, such as grafts of surrounding tissues. Examples of wounds not readily treatable with staples or suturing include large, deep, open wounds; decubitus ulcers; ulcers resulting from chronic osteomyelitis; and partial thickness burns that subsequently develop into full thickness burns.

As a result of these and other shortcomings of mechanical closure devices, methods and apparatus for healing wounds by applying continuous negative pressures have been developed. When applied over a sufficient area of the wound, negative pressures have been found to promote the migration toward the wound of epithelial and subcutaneous tissues. In practice, the application of negative pressure to a wound is commonly referred to as negative pressure wound therapy (NPWT). NPWT typically involves mechanical-like contraction of the wound with simultaneous removal of excess fluid. In this manner, NPWT therapy augments the body's natural inflammatory process while alleviating many of the known intrinsic side effects, such as the production of edema caused by increased blood flow absent the necessary vascular structure for proper venous return.

While negative pressure wound therapy has been highly successful in the promotion of wound closure, healing many wounds previously thought largely untreatable, some difficulty remains. Because the inflammatory process is very unique to the individual patient, even the addition of negative pressure wound therapy does not result in a fast enough response, especially during the occlusion and initial cleanup and rebuilding stages, for adequate healing of some wounds. Also, many of these wounds include decubitus and venous stasis ulcers to the lower extremities, especially the foot. Closure of these wounds has been difficult and often times impossible using traditional techniques, such as skin grafting, sharp debridement, or combinations thereof. Failure to close these wounds, which have often been present for several years, can lead to necrotizing of the tissue, and in many cases amputation of the extremity. Use of negative pressure wound therapy has proven highly successful in closing these wounds. However, treatment of the lower extremities with negative pressure wound therapy, especially to wounds of the foot and heal can be difficult, considering the nature of the location of the wound. Particular concern arises with maintaining the dressing on the extremity, especially in light of the frequent movement of the foot, and friction often associated with foot coverings, including socks, stockings, and shoes. Of particular concern, is the ability to maintain a negative pressure at the wound site when the dressing is in place, as air leaks may occur during movement of the foot, which can adversely affect the therapy being administered.

Moreover, the FDA has recently reported adverse events resulting from the use of NWPT. See, www.fda.gov/consumer, FDA Consumer Health Information, December 2009, Negative Pressure Wound Devices Draw FDA Notice, Advice. Page 2, column 1, of the FDA article, mentions cases of infections from original open infected wounds worsening due to pieces of dressing that remained in a wound, and of injury from foam dressing pieces and foam sticking to the tissues or clinging to wounds.

B. Description of the Related Art

U.S. Pat. No. 5,829, 442, assigned to Medical Device Concepts , discloses an adhesive composition for use in wound dressings comprising hot melt adhesives, wherein said composition is a mixture of 2 acrylic polymers, one of a low molecular weight (MW) and the other of a medium MW. Hydrocarbon tackfiers are also present (e.g. Foral). A representative composition is 17% low MW acrylic, 67% medium MW acrylic and 16% tackfier. Hydrocolloids are not disclosed

US application 2004/0096489, assigned to Molnlycke, discloses skin friendly adhesives and wound dressings comprising acrylate adhesives and tacky hot melt adhesives (e.g. Dispomelt) and carboxymethyl cellulose. Acrylics are not disclosed in US 2004/0096489.

2010/0022961 discloses a dressing composition containing a hydrocolloid material, such as calcium carboxymethylcellulose (“CMC”), pectin, gelatin, high molecular weight carbowax, carboxypolymethylene, polyacrylate, polyvinyl alcohol, and polyvinyl pyrrolidone; a tackifier, such as a hydrocarbon resin; a hot melt acrylic; an elastomer such as a styrene-olefin-styrene compound, polyisobutylene, natural rubber, silicone rubber, acrylonitrile rubber, and polyurethane rubber. Also, said composition may include an extender, such as mineral oil or paraffin oil. Said composition is described as being useful in ostomy applications.

Further, typical NPWT dressings are exemplified by the V.A.C. GranuFoam Dressings available from KCI. Smith and Nephew also markets foam and gauze dressings under the respective tradenames of RENASYS-F and RENASYS -G.

Now, the NPWT dressing of the present invention provides clinicians with a dressing that overcomes the problems in the art. For example, foam or gauze are typically placed on the wound bed and taped in place with a non absorptive polyurethane film. On the other hand, the Applicants' dressing, unlike said polyurethane film, when placed atop said foam or gauze, is absorptive. The Applicants' dressing keeps the foam or gauze in place and absorbs wound fluid and perspiration to maintain the proper environment for wound healing. Also, Applicants' incorporation of acrylic facilitates adherance of the dressing and obviates the need for additional taping to secure the dressing to the patient's skin.

### SUMMARY OF THE INVENTION

The present invention relates to a wound dressing composition suitable for use in a negative pressure wound therapy application comprising:

a. 20-60% by weight of a hydrocolloid and mixtures thereof;

b. 2-30% by weight of a hot melt acrylic adhesive and mixtures thereof;

c. 5-40% by weight of a tackifier and mixtures thereof;

d. 5-30% by weight of an elastomeric binder and mixtures thereof;

e. 5-30% by weight of an extender and mixtures thereof.

The following terms have the following usages and meanings herein: “Negative Pressure Wound Therapy” (NPWT) is a therapeutic technique used to promote healing in acute or chronic wounds. A vacuum source is used to create sub atmospheric pressure in a local wound environment. NPWT seals the wound to prevent dehiscence with a gauze or foam filler dressing, a drape, and a vacuum source that applies negative pressure to the wound bed with a tube threaded through the dressing. The vacuum may be applied continuously or intermittently, depending upon the type of wound being treated.

“Acrylic acid” is a highly corrosive unsaturated carboxylic acid used to produce acrylates, e.g. acrylic esters such as methyl acrylate, butyl acrylate, ethyl acrylate, and 2-ethylhexyl acrylate. Acrylic acid is also used to produce polyacrylic acid, or crosslinked polyacrylic acid compounds.

### DETAILED DESCRIPTION OF THE INVENTION

The present invention relates to a wound dressing composition suitable for use in a negative pressure wound therapy application comprising:

a. 20-60% by weight of a hydrocolloid and mixtures thereof;

b. 2-30% by weight of a hot melt acrylic adhesive and mixtures thereof;

c. 5-40% by weight of a tackifier and mixtures thereof;

d. 5-30% by weight of an elastomeric binder and mixtures thereof;

e. 5-30% by weight of an extender and mixtures thereof.

The present invention also relates to a wound dressing composition suitable for use in a negative pressure wound therapy application comprising:

a. 30-55% by weight of a hydrocolloid and mixtures thereof;

b. 3-20% by weight of a hot melt acrylic adhesive and mixtures thereof;

c. 10-40% by weight of a tackifier and mixtures thereof;

d. 10-25% by weight of an elastomeric binder and mixtures thereof;

e. 5-20% by weight of an extender and mixtures thereof.

The present invention also relates to a wound dressing composition suitable for use in a negative pressure wound therapy application comprising:

a. 33-51% by weight of a hydrocolloid and mixtures thereof;

b. 6-14% by weight of a hot melt acrylic adhesive and mixtures thereof;

c. 15-40% by weight of a tackifier and mixtures thereof;

d. 10-20% by weight of an elastomeric binder and mixtures thereof;

e. 5-10% by weight of an extender and mixtures thereof.

The present invention further relates to a wound dressing composition suitable for use in a negative pressure wound therapy application comprising:

a. 33% by weight of a hydrocolloid, wherein said hydrocolloid is carboxymethylcellulose,

b. 6% by weight of a hot melt acrylic adhesive;

c. 38.5% by weight of a tackifier, wherein said tackfier is a hydrocarbon tackifier;

d. 15% by weight of an elastomeric binder, wherein said elastomeric binder is an olefinic copolymer;

e. 7.5% by weight of an extender, wherein said extender is mineral.

Hydrocolloids

Hydrocolloids are known in the wound care art. Hydrocolloids useful in the practice of the present invention include, but are not limited to, water absorbing and/or water swellable material such as carboxymethylcellulose, pectin, gelatin, high molecular weight carbowax, carboxypolymethylene, carboxymethyl starches, alginates, carrageenan, gelatine, citrus pectin, powdered pectin, synthetic or natural gums, such as gum guar, gum arabic, locust bean gum, karaya and mixtures thereof. In an embodiment of the invention, the preferred hydrocolloids are carboxymethylcellulose, alginates and pectin. In another embodiment of the invention, carboxymethylcellulose (CMC) is preferred.

Elastomeric Binders

Elastomeric binders useful in the practice of the present invention include, but are not limited to, diblock, triblock, or multiblock elastomeric copolymers such as olefinic copolymers such as styrene-isoprene-styrene, styrene-butadiene-styrene, styrene-ethylene/butylene-styrene, or styrene-ethylene/propylene-styrene, such as those available from the Shell Chemical Company, under the trade designation KRATON® elastomeric resin; polyurethanes, such as those available from E. I. Du Pont de Nemours Co., under the trade name LYCRA® polyurethane; polyamides, such as polyether block amides available from Ato Chemical Company, under the trade name PEBAX® polyether block amide; or polyesters, such as those available from E. I. Du Pont de Nemours Co., under the trade name HYTREL® polyester; natural rubbers, silicone rubber, polyisobutylene rubber, and acrylonitrile rubber. In an embodiment of the invention, mixtures of elastomeric binders may be used. In an exemplary embodiment, the KRATON® olefinic copolymers are preferred.

Tackifiers

Tackifiers include, but are not limited to, pine derived rosins (gum rosin, wood rosin, tall oil rosin) and hydrogenated rosins, hydrocarbons and hydrogenated hydrocarbon resins such as C5 aliphatic resins, C9 aromatic resins, and C5/C9 aliphatic/aromatic resins; pure monomers, hydrogenated pure monomers, and water based dispersions. Representative tackifiers, known by their tradenames are FORAL® 85 and ARKON® DP115. FORAL® 85 is a hydrocarbon tackifier. ARKON® P115 is a hydrogenated hydrocarbon tackfier. In an embodiment of the invention, mixtures of tackifiers may be used. In a further embodiment of the invention, FORAL® 85 and ARKON® DP115 are preferred.

Hot Melt Acrylic Adhesives

In an exemplary embodiment, the hot melt acrylic adhesive may be a 100% solid acrylic polymer available from Franklin Adhesives & Polymers, a division of Franklin International, under the Tradename Acrynax. In an embodiment of the invention, mixtures of acrylic adhesives may be used.

Extenders

Extenders include, but are not limited to, paraffin oil and mineral oil. The extender may also be a material that functions as a plasticizer, particularly in combination with the elastomeric binder. Such plasticizers include glycerin (glycerol), sorbitol,and triethylene glycol. In an embodiment of the invention, mixtures of extenders may be used. In a further embodiment, mineral oil is preferred.

The composition of the present invention is prepared by blending all ingredients using techniques known to those skilled in the art.

Active Ingredients

One or more active ingredients may be blended with the compositions of the present invention.

Non limiting examples of active ingredients that may be blended with the composition of the present invention include:

1. Circulatory drugs, such as, for example, organic nitrates, such as nitroglycerol isosorbide dinitrate; procainamide; thiazides; dihydropyridines, such as nifedipine or nicardipine; beta blockers, such as timolol or propranolol; ACE inhibitors, such as enalapril, captopril or lisinopril; or alpha-2 blockers, such as clonidine or prazosine.

2. Androgenic steroids, such as testosterone, methyl testosterone or fluoximesterone.

3. Oestrogens, such as oestradiol esters, oestradiol propionate, 17-.beta.-oestradiol, 17-.beta.-oestradiol valerate, oestrone, mestranol, oestriol, 17.beta.-ethynyl-oestradiol or diethylstilboestrol.

4. Progestagenic hormones, such as progesterone, 19-Nor-progesterone, norethisterone, norethisterone acetate, melengoestrol, chlormadinone, ethisterone, medroxy-progesterone acetate, hydroxyprogesterone caproate, ethynodiol diacetate, 17-.alpha.-hydroxyprogesterone, norgestrel and others.

5. Active ingredients having an action on the central nervous system, such as, for example, sedatives, hypnotics, anxiolytics, antidepressants, analgesics and anaesthetics, such as buprenorphine, naloxone, haloperidol, flufenacin, barbitals, lidocaine, mepivacaine, fentanyl, suventanil or nicotine.

6. Agents for treatment of Parkinson's disease, such as selegiline salts or seligiline base, bromocriptine, lisuride and others.

7. Anti-inflammatory active ingredients, such as hydrocortisone, cortisone, dexamethasone, triamcinolone, prednisolone, ibuprofen, naproxen, fenoprofen, flurbiprofen, indoprofen, ketoprofen, piroxicam, diflunisal and others.

8. Antihistamines, such as dimenhydrinate, perfenacin, prometacin, terfenadin, loratidine and others.

9. Active ingredients having an action on the respiratory tract, such as theophylline or beta₂-adrenergic agonists, such as albuterol, terbutalin, fenoterol, salbutamol and others.

10. Sympathicomimetics, such as dopamine, phenylpropanolamine, phenylephrine and others.

11. Antimuscarines, such as atropine, scopolamine, homatropine, benzatropine and others.

12. Dermatological active ingredients, such as vitamin A, cyclosporin, dexpanthenol and others.

13. Prostaglandins, such as prostaglandin E1, prostaglandin E2, prostaglandin F2 and analogs.

14. Antioestrogens, such as tamoxifen and 3-hydroxy- and 4-hydroxytamoxifen.

15. Antimigraine agents, such as dihydroergotamine and pizotyline.

16. Antiulcer agents, such as misoprostol, omeprazole, enprostil or ranitidine.

17. Vitamins, Minerals and Botanicals—Vitamins useful in the practice of the present invention include A, D, E, K, B and C. Minerals include, but are not limited to, Calcium, Chromium, Selenium, Iron, Manganese, Magnesium, Potassium, Sodium, Phosphorus, Zinc and Copper. Botanicals include, but are not limited to arnica, astrala gus, aniseed, barberry, blackberry, black cohosh, borage, broom, bearberry, birch, blood root, calendual, cayenne, chamomile, coltsfoot, celery seeds, cascara sagrada, chickweed, corn silk, catmint, daisy, dandelion, dill, Echinacea, elder, eucalyptus, fennel, fenugreek, feverfew, figwort, garlic, ginger, golden seal, gingko biloba, ginseng, hawthorn, hops, horehound, horse chestnut, hyssop, hydrangea, irish moss, Iceland moss, juniper berries, kola, lavendar, linden , licorice, lobelia, ma huang, marshmallow, mugwort, mustard, milk thistle, myrrh, nettle, oak bark, oats, parsley, passion flower, peppermint, periwinkle, prickly ash, poke, pennyroyal, raspberry, red clover, rhubarb root, rosemary, sarsaparilla, saw palmetto, senna, slippery elm, St John's wort, sweet violet, skull cap, snake root, tea tree, thyme, turmeric, valerian, western hemlock, wild carrot, wild yam, wild lettuce, willow, witch hazel, wormwood, yarrow and yellow dock.

Exemplary embodiments of the present invention have been described. Persons of ordinary skill in the art will appreciate that variations may be made without departure from the scope and spirit of the invention. This true scope and spirit is defined by the appended claims, interpreted in light of the foregoing.

We claim:

1. A wound dressing composition suitable for use in a negative pressure wound therapy application comprising:
a. 20-60% by weight of a hydrocolloid and mixtures thereof;
b. 2-30% by weight of a hot melt acrylic adhesive and mixtures thereof;
c. 5-40% by weight of a tackifier and mixtures thereof;
d. 5-30% by weight of an elastomeric binder and mixtures thereof;
f. 5-30% by weight of an extender and mixtures thereof.

2. A wound dressing composition suitable for use in a negative pressure wound therapy application comprising:
a. 30-55% by weight of a hydrocolloid and mixtures thereof;
b. 3-20% by weight of a hot melt acrylic adhesive and mixtures thereof;
c. 10-40% by weight of a tackifier and mixtures thereof;
d. 10-25% by weight of an elastomeric binder and mixtures thereof;
e. 5-20% by weight of an extender and mixtures thereof

3. A wound dressing composition suitable for use in a negative pressure wound therapy application comprising:
a. 33-51% by weight of a hydrocolloid and mixtures thereof;
b. 6-14% by weight of a hot melt acrylic adhesive and mixtures thereof;
c. 15-40% by weight of a tackifier and mixtures thereof;
d. 10-20% by weight of an elastomeric binder and mixtures thereof;
e. 5-10% by weight of an extender and mixtures thereof.

4. A wound dressing composition suitable for use in a negative pressure wound therapy application comprising:
a. 33% by weight of a hydrocolloid, wherein said hydrocolloid is carboxymethylcellulose;
b. 6% by weight of a hot melt acrylic adhesive;
c. 38.5% by weight of a tackifier, wherein said tackfier is a hydrocarbon tackifier;
d. 15% by weight of an elastomeric binder, wherein said elastomeric binder is an olefinic copolymer;
e. 7.5% by weight of an extender, wherein said extender is mineral.

5. A wound dressing composition suitable for use in a negative pressure wound therapy application comprising:
a. 20-60% by weight of a hydrocolloid and mixtures thereof;
b. 2-30% by weight of a hot melt acrylic adhesive and mixtures thereof;
c. 5-40% by weight of a tackifier and mixtures thereof;
d. 5-30% by weight of an elastomeric binder and mixtures thereof;
e. 5-30% by weight of an extender and mixtures thereof, wherein further, said composition comprises one or more active ingredients.
